(19) United States
(12) Patent Application Publication
(10) Pub. No.: US 2008/0255207 A1
(43) Pub. Date: Oct. 16, 2008

(54) 5-Thioxo-1,5-Dihydro-2H-Pyrrol-2-One Derivatives As Liver X Receptor Modulators

(75) Inventors: Patrik Holm, Pargas (FI); Lanna Li, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/813,467

(22) PCT Filed: Jan. 9, 2006
(86) PCT No.: PCT/SE2006/000030

§ 371 Date: Jul. 6, 2007

(30) Foreign Application Priority Data
Jan. 10, 2005 (SE) 0500058-3

(51) Int. Cl. A61K 31/4439; C07D 401/12

(52) U.S. Cl. 514/343; 546/278.7

(57) ABSTRACT

The present invention relates to certain novel compounds of the Formula (I) to processes for preparing such compounds, to their the utility in modulation of nuclear hormone receptors Liver X Receptor (LXR) α(NR1H3) and/or β(NR1H2) and in treating clinical conditions including cardiovascular diseases such as atherosclerosis; inflammatory diseases, Alzheimer's disease, lipid disorders (dyslipidemias) whether or not associated with insulin resistance, type 2 diabetes and other manifestations of the metabolic syndrome, to methods for their therapeutic use and to pharmaceutical compositions containing them.

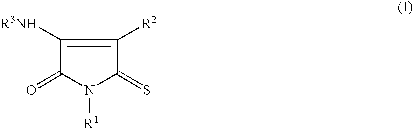

### FIELD OF THE INVENTION

The present invention relates to certain novel, substituted 5-thioxo-1,5-dihydro-2H-pyrrol-2-one, to processes for preparing such compounds, to their the utility in modulation of nuclear hormone receptors liver X Receptor (LXR) α(NR1H3) and/or β(NR1H2) and in treating clinical conditions including cardiovascular diseases such as atherosclerosis; inflammatory diseases, Alzheimer's disease, lipid disorders (dyslipidemias) whether or not associated with insulin resistance, type 2 diabetes and other manifestations of the metabolic syndrome, to methods for their therapeutic use and to pharmaceutical compositions containing them.

### BACKGROUND OF THE INVENTION

Abnormalities of cholesterol and fatty acid homeostasis, that are reflected as diverse dyslipidemias, are causal of atherosclerosis and consequently cardiovascular disease (CVD). This disease is one of the major health problems in industrialized countries and is reaching the same prevalence in adults in developing nations. Most studies show that statins reduce-low density lipoproteins (LDL) cholesterol by 25-30% and the relative risk of coronary events by approximately 30%. While this beneficial effect is significant, effectively 70% of the treated cohort remains with unchanged risk. This has prompted intense research in order to identify other common abnormalities of lipid metabolism that if efficiently treated could improve the results of current CVD therapy.

The nuclear hormone receptors LXR α and β use oxysterols as natural ligands. They appear to act as cholesterol sensors with target genes that are required for cholesterol efflux from macrophages, like ATP binding casette transporter A1 (ABCA1) and apoE as well as gene products, like cholesterol ester transferase protein (CETP) and phospholipid transport protein (PLTP), that are required for the function of high density lipoprotein (HDL) in the reverse cholesterol transport. In addition, LXR upregulates lipoprotein lipase in liver and macrophages, a function that may stimulate fatty acid uptake and very low density lipoprotein (VIDL) remodeling. In the liver, TYR ligands seem to stimulate the hepatobiliary secretion of cholesterol, a pathway controlled by the ABCG5 and ABCG8. The same cholesterol transporters appear to reduce cholesterol absorption in enterocytes, therefore influencing total body cholesterol balance. These effects of LXR stimulation could explain its remarkable anti-atherosclerotic properties observed in several animal models.

Recently the synthetic LXR ligands GW3965 (Glaxo) and T-0901317 (Tularik) were reported to increase glucose tolerance in fat fed obese mouse which was interpreted to result from reduced hepatic gluconeogenesis and increased glucose uptake in adipocytes Lafitte B A et al. (Proc Natl Acad Sci USA. 2003 Apr. 29; 100(9):5419-24). Activation of LXR's improves glucose tolerance through coordinated regulation of glucose metabolism in liver and adipose tissue.

WO00/21927 discloses pyrrole-2,5-diones which are GSK-3 inhibitors and claimed to be useful in the treatment of dementias such as Alzheimer's disease, manic depression and diabetes. There is no suggestion that these compounds have activity as LXR modulators.

The term “LXR modulator” as used herein, means a small molecule that modulates the biological activities of LXR α and/or LXR β. More specifically, such an LXR modulator either enhances or inhibits the biological activities of LXR. If such a modulator partially or completely enhances the biological activities of LXR, it is a partial or full LXR agonist, respectively. It is the object of the present invention to provide LXR modulators. Another object of this invention is to provide LXR modulator compounds being LXR agonists.

### DESCRIPTION OF THE INVENTION

According to a first aspect of the present invention there is provided a compound of Formula I

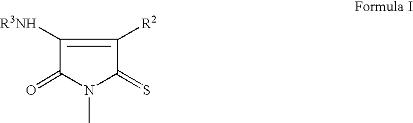

wherein:
R¹ is selected from phenyl(1-4C)alkyl wherein the phenyl is optionally substituted by (1-4C)alkoxycarbonyl or a group of formula NR^{a}R^{b} in which R^{a} and R^{b} independently represent H or (1-4C)alkyl; heteroaryl(1-4C)alkyl wherein the heteroaryl is optionally substituted by (1-4C)alkyl or a group of formula NR^{a}R^{b} in which R^{a} and R^{b} independently to represent H or (1-4C)alkyl; or a (1-6C)alkyl group which is optionally-substituted by one or more of the following: fluoro, (1-4C)alkoxycarbonyl, (1-3C)alkylthio or (1-3C)alkoxy optionally substituted by one or more fluoro;
R² is phenyl;
R¹ is selected from phenyl, indolyl or benzofuranyl each optionally substituted by one or more of the following: (1-3C)alkanoyl, (1A4C)alkoxy optionally substituted by one or more fluoro; (1-3C)alkylthio; or a group of formula NR^{a}R^{b} in which R^{a} and R^{b} independently represent H, (1-3C)alkyl or (1-3C)alkanoyl or R^{a} and R^{b} together with the nitrogen atom to which they are attached represent morpholino;
or a pharmaceutically acceptable salt or solvate thereof, or a solvate of such a salt.

The term heteroaryl means pyridyl, furyl or isoxazolyl each of which is optionally substituted by one or more of the following: (1-4C)alkyl or a group of formula NR^{a}R^{b} in which R^{a} and R^{b} independently represent H or (1-4C)alkyl.

Further values of R¹, R², and R³ in compounds of formula I now follow. It will be understood that such values may be used where appropriate with any of the definitions, claims or embodiments defined hereinbefore or hereinafter.

In a first group of compounds of formula I

R¹ is selected from methyl, ethyl, propyl, butyl, 2,2,2-trifluoroethyl, benzyl, 2-methoxyethyl, 3-pyridylmethyl, 4-pyridylmethyl or 6-amino-3-pyridylmethyl;
R² is phenyl;
R³ is selected from 4-methoxyphenyl, 4-difluoromethoxyphenyl or 4-morpholinophenyl;

In a second group of compounds of formula I

R¹ is selected from methyl, ethyl, 2,2,2-trifluoroethyl, benzyl, 3-pyridylmethyl or 6-amino-3-pyridylmethyl;
R² is phenyl;
R³ is selected from 4-methoxyphenyl, 4-difluoromethoxyphenyl or 4-morpholinophenyl;

In a fourth group of compounds of formula I

R¹ is selected from ethyl, 2,2,2-trifluoroethyl, benzyl, 3-pyridylmethyl, 6-amino-3-pyridylmethyl;
R² is phenyl;
R³ is selected from 4-methoxyphenyl, 4-difluoromethoxyphenyl or 4-morpholinophenyl;

In a fifth group of compounds of formula I

R¹ is selected from methyl, ethyl, 2,2,2-trifluoroethyl, 2-pyridylmethyl, 3-pyridylmethyl, 4-pyridylmethyl;
R² is phenyl;
R³ is selected from 4-methoxyphenyl;

In a sixth group of compounds of formula I

R¹ is selected from 2-methoxyethyl or 6-amino-3-pyridylmethyl;
R² is phenyl;
R³ is selected from 4-methoxyphenyl or 4-difluoromethoxyphenyl;

The compounds of formula I have activity as medicaments. In particular the compounds of formula I are LXR agonists.

A specific compound of the invention is 1-[(6-aminopyridin-3-yl)methyl]-3-{[4-(difluoromethoxy)phenyl]amino}-4-phenyl-5-thioxo-1,5-dihydro-2H-pyrrol-2-one or a pharmaceutically acceptable salt or solvate thereof, or a solvate of such a salt.

Certain compounds of the present invention may exist as tautomers. It is to be understood that the present invention encompasses all such tautomers.

### Methods of Preparation

The compounds of the invention may be prepared as outlined below. However, the invention is not limited to these methods. The compounds may also be prepared as described for structurally related compounds in the prior art. The reactions can be carried out according to standard procedures or as described in the experimental section.

Compounds of formula I may be prepared by reacting a compound of formula II

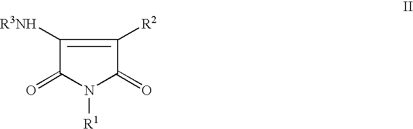

in which R¹, R² and R³ are as previously defined with a sulphurating agent, for example Lawesson's reagent, optionally in the presence of an inert organic liquid for example an aromatic hydrocarbon, e.g. toluene, or an organic liquid for example an ether, e.g. dioxane at a temperature in the range of 0° C. to 200° C. Compounds of formula I may be prepared using an approximately molar equivalent of the sulphurating agent.

Compounds of formula II may be prepared by reacting a compound of formula III

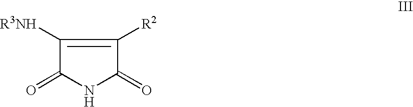

in which R² and R³ are as previously defined with a compound of formula IV

R¹OH IV

in which R¹ is as previously defined in the presence of a dialkyl azodicarboxylate, for example diethyl azodicarboxylate, and a phosphine, for example triphenylphosphine, optionally in the presence of an inert organic liquid for example an ether e.g. tetrahydrofuran at a temperature in the range of 0° C. to 200° C.

Compounds of formula II may also be prepared by reacting a compound of formula V

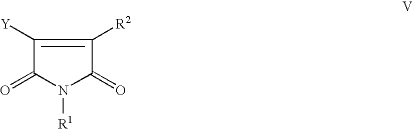

is in which R¹ and R² are as previously defined and Y is a leaving group for example halo e.g. Cl, Br or I with a compound of formula VI

R³NH₂ VI

in which R³ is as previously defined optionally in the presence of an inert organic liquid for example dimethylformamide and optionally in the presence of a base for example potassium carbonate at a temperature in the range of 0° C. to 250° C.

Compounds of formula III may be prepared by reacting a compound of formula VII

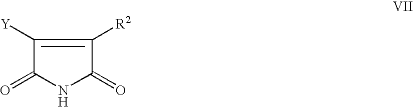

in which R² is as previously defined and Y is a leaving group for example halo eg Cl, Br or I with a compound of formula VI

R³NH₂ VI

in which R³ is as previously defined optionally in the presence of an inert organic liquid for example dimethylformamide and optionally in the presence of a base for example triethylamine at a temperature in the range of 0° C. to 250° C.

Compounds of formula IV and VI are commercially available or may be prepared by methods known to those skilled in the art.

Compounds of formula V may be prepared by reacting a compound of formula VIII

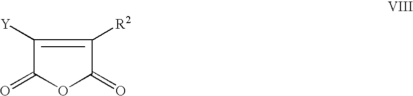

in which R² is as previously defined and Y is a leaving group for example halo eg Cl, Br or I with a compound of formula IX

R¹NH₂ IX

in which R¹ is as previously defined optionally in the presence of an organic liquid, for example glacial acetic acid at a temperature in the range of 0° C. to 200° C.

Compounds of formula V may also be prepared by reacting a compound of formula VII with a compound of formula XII

R¹L XII

in which R¹ is as previously defined and L is a leaving group for example halo eg bromo in the presence of an inert organic liquid for example dimethylformaride and optionally in the presence of a base for example potassium carbonate at a temperature in the range of −78° C. to 200° C.

Compounds of formula VIII may be prepared by reacting a compound of formula X

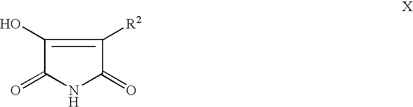

in which R² is as previously defined with a halogenating agent for example oxalyl chloride optionally in the presence of an inert organic liquid for example dichloromethane and optionally in the presence of a catalytic amount of dimethylformamide at a temperature in the range of 0° C. to 200° C.

Compounds of formula VIII may be prepared by reacting a compound of formula XI

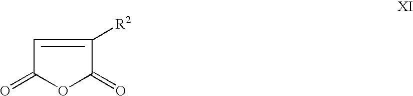

in which R² is as previously defined with a halogenating agent for example thionyl chloride optionally in the presence of an inert organic liquid for example dichloromethane and optionally in the presence of a base for example pyridine at a temperature in the range of 0° C. to 200° C.

Compounds of formula IX, X, A, and XII are commercially available or may be prepared by methods known to those skilled in the art.

Certain compounds of formula III and V are useful intermediates in the preparation of compounds of formula I and are believed to be novel and are claimed herein as useful intermediates in the preparation of compounds of formula I.

The compounds of the invention may be isolated from their reaction mixtures using conventional techniques.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

The expression “inert organic liquid” refers to a liquid that does not react with the starting materials, reagents, intermediates or products in a manner that adversely affects the yield of the desired product.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable salt or solvate thereof, or a solvate of such a salt, in a pharmaceutically acceptable dosage form. Depending upon the disorder and patient to be treated and the route of administration, the compositions may be administered at varying doses.

Suitable daily doses of the compounds of the invention in therapeutical treatment of humans are about 0.0001-100 mg/kg body weight, preferably 0.01-10 mg/kg body weight.

Oral formulations are preferred particularly tablets or capsules which may be formulated by methods known to those skilled in the art to provide doses of the active compound in the range of 0.007 mg to 700 mg for example 1 mg, 3 mg, 5 mg, 10 mg, 25 mg, 50 mg, 100 mg and 250 mg.

According to a further aspect of the invention there is thus provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula I are useful for normalization of cholesterol homeostasis, decreasing intestinal cholesterol absorption, improving reverse cholesterol transport, improving HDL functionality, increasing HDL-cholesterol levels, decreasing LDL-cholesterol levels, decreasing cholesterol content of apoB-containing lipoproteins, stimulating cholesterol efflux from vascular cells and/or decreasing the inflammatory response of vascular cells. As a consequence of these properties the compounds of formula I are expected to have anti-atherosclerotic effects.

The compounds of formula I are useful in the prevention or treatment of cardiovascular disease in a mammal, particularly a human. The compounds of formula I are useful in the prevention or treatment of atherosclerosis in a mammal, particularly a human. Cardiovascular disease includes but is not limited to conditions associated with atherosclerosis, arteriosclerosis, hypercholesterolemia, and other kinds of dyslipidemia that increase the risk for cardiovascular disease. In particular the compounds of formula I are useful in the treatment or prevention of cardiovascular disease, especially those involving atherosclerosis and hypercholesterolemia.

The compounds of formula I also serve to prevent lipid accumulation in, or remove lipids from, tissue deposits such as atherosclerotic plaques or xanthomas in a patient with atherosclerotic disease manifest by clinical signs such as angina, claudication, bruits, one that has suffered a mycardial infarction or transient ischemic attack, or one diagnosed by angiography, sonography or MRI.

The compounds of formula I also serve to prevent or reduce the risk of developing atherosclerosis, as well as for halting or slowing the progression of atherosclerotic disease once it has become clinically evident, comprising the administration of a prophylactically or therapeutically effective amount, as appropriate, of a compound of formula I to a mammal, including a human, who is at risk of developing atherosclerosis or who already has atherosclerotic disease.

Atherosclerosis encompasses vascular diseases and conditions that are recognized and understood by physicians practicing in the relevant fields of medicine. Atherosclerotic cardiovascular disease including restenosis following revascularization procedures, coronary heart disease (also known as coronary artery disease or ischemic heart disease), cerebrovascular disease including multi-infarct dementia, and peripheral vessel disease including erectile dysfunction are all clinical manifestations of atherosclerosis and are therefore encompassed by the terms “atherosclerosis” and “atherosclerotic disease”.

The present compounds of formula I are also useful for the prophylaxis and/or treatment of clinical conditions associated with atherosclerosis such as inherent or induced hypercholesterolemia as well as inherent or induced reduced sensitivity to insulin (insulin resistance syndrome also known as metabolic syndrome) and associated metabolic disorders. These clinical conditions will include, but will not be limited to, general obesity, abdominal obesity, arterial hypertension, hyperinsulinaemia, hyperglycaemia, type 2 diabetes and the dyslipidaemia characteristically appearing with insulin resistance. This dyslipidaemia, also known as the atherogenic lipoprotein profile, is characterised by moderately elevated non-esterified fatty acids, elevated VLDL triglyceride rich particles, high Apo B levels, low HDL levels associated with low apoAI levels in the presence of small, dense, LDL particles, phenotype B.

The compounds of formula I are expected to be useful in treating patients with combined or mixed hyperlipidemias and dyslipidemias, especially low HDL levels with or without other manifestations of the metabolic syndrome.

Treatment with the compounds of formula I are expected to lower the cardiovascular morbidity and mortality associated with atherosclerosis due to their antidyslipidaemic as well as antiinflammatory properties. The cardiovascular disease conditions include macro-angiopathies of various internal organs causing myocardial infarction, congestive heart failure, cerebrovascular disease and peripheral arterial insufficiency of the lower extremities. The insulin sensitizing effect of the compounds of formula I is also expected to prevent or delay the development of type 2 diabetes from the metabolic syndrome and diabetes of pregnancy. Therefore the development of long-term complications associated with chronic hyperglycaemia in diabetes mellitus such as the micro-angiopathies causing renal disease, retinal damage and peripheral vascular disease of the lower limbs are expected to be delayed.

The compounds of formula I may also be useful for the prevention or treatment of inflammation and neurodegenerative diseases or neurological disorders. Accordingly, this invention also provides a method for preventing or treating inflammation in the CNS, and a method for preventing or treating neurodegenerative diseases or disorders characterized by neuron degeneration, neuron injury or impaired plasticity or inflanmnation in the CNS. The neurodegenerative diseases or conditions characterized by neuron degeneration and inflammation will include but will not be limited to stroke, Alzheimer's disease, fronto-temporal dementias (taupathies), peripheral neuropathy, Parkinson's disease, dementia with Lewy bodies, Huntington's disease, amyotrophic lateral sclerosis and multiple sclerosis.

The compounds of formula I are useful in preventing or treating inflammatory conditions or diseases. These diseases or conditions will include but will not be limited to atherosclerotic diseases such as angina pectoris and myocardial infarction as well as inflammatory bowel diseases or conditions such as Crohn's disease, ulcerative colitis and distal proctitis. Compounds of formula I may also be used in other inflammatory conditions of the lung including asthma, adult respiratory distress syndrome, chronic obstructive pulmonary disease and pneumonia bronchitis.

Furthermore the compounds of formula I may be useful in treatment of various conditions outside the cardiovascular system whether or not associated with insulin resistance, like polycystic ovarian syndrome, obesity and cancer.

The present invention provides a method of treating and/or preventing dyslipidemias, the insulin resistance syndrome and/or metabolic disorders (as defined above) comprising the administration of a compound of formula I to a mammal (particularly a human) in need thereof.

The present invention provides a method of treating and/or preventing type 2 diabetes comprising the administration of an effective amount of a compound of formula I to a mammal (particularly a human) in need thereof.

The present invention provides a method of treating and/or preventing cardiovascular disease comprising the administration of an effective amount of a compound of formula I to a mammal (particularly a human) in need thereof.

The present invention provides a method of treating and/or preventing atherosclerosis comprising the administration of an effective amount of a compound of formula I to a mammal (particularly a human) in need thereof.

The present invention provides a method of treating and/or preventing hypercholesterolemia comprising the administration of an effective amount of a compound of formula I to a mammal (particularly a human) in need thereof.

The present invention provides a method of treating and/or preventing conditions associated with a need for improving reverse cholesterol transport comprising the administration of an effective amount of a compound of formula I to a mammal (particularly a human) in need thereof.

The present invention provides a method of treating and/or preventing conditions associated with a need for decreasing intestinal cholesterol absorption comprising the administration of an effective amount of a compound of formula I to a mammal (particularly a human) in need thereof.

The present invention provides a method of treating and/or preventing conditions associated with a need for increasing HDL-cholesterol levels comprising the administration of an effective amount of a compound of formula I to a mammal (particularly a human) in need thereof.

The present invention provides a method of treating and/or preventing conditions associated with a need for decreasing LDL-cholesterol levels comprising the administration of an effective amount of a compound of formula I to a mammal (particularly a human) in need thereof.

The present invention provides a method of treating and/or preventing inflammatory conditions comprising the administration of an effective amount of a compound of formula I to a mammal (particularly a human) in need thereof.

The present invention provides a method of treating and/or preventing Alzheimer's disease comprising the administration of an effective amount of a compound of formula I to a mammal (particularly a human) in need thereof.

The present invention provides a method of treating and/or preventing arteriosclerosis comprising the administration of an effective amount of a compound of formula I to a mammal (particularly a human) in need thereof.

The present invention provides a method of treating and/or preventing conditions associated with a need for improving HDL function comprising the administration of an effective amount of a compound of formula I to a mammal (particularly a human) in need thereof.

The present invention provides a method of treating and/or preventing hyperlipidemic conditions comprising the administration of an effective amount of a compound of formula I to a mammal (particularly a human) in need thereof.

In a further aspect the present invention provides the use of a compound of formula I as a medicament.

In a further aspect the present invention provides the use of a compound of formula I in the manufacture of a medicament for the treatment and/or prohylaxis of dyslipidemic conditions.

In a further aspect the present invention provides the use of a compound of formula I in the manufacture of a medicament for the treatment and/or prohylaxis of insulin resistance syndrome and/or metabolic disorders.

In a further aspect the present invention provides the use of a compound of formula I in the manufacture of a medicament for the treatment and/or prophylaxis of cardiovascular disease.

In a further aspect the present invention provides the use of a compound of formula I in the manufacture of a medicament for the treatment and/or prophylaxis of atherosclerosis.

In a further aspect the present invention provides the use of a compound of formula I in the manufacture of a medicament for the treatment and/or prophylaxis of hypercholesterolemia.

In a further aspect the present invention provides the use of a compound of formula I in the manufacture of a medicament for the treatment and/or prophylaxis of conditions associated with a need for improving reverse cholesterol transport.

In a further aspect the present invention provides the use of a compound of formula I in the manufacture of a medicament for the treatment and/or prophylaxis of conditions associated with a need for decreasing intestinal cholesterol absorption.

In a further aspect the present invention provides the use of a compound of formula I in the manufacture of a medicament for the treatment and/or prophylaxis of conditions associated is with a need for increasing LDL-cholesterol levels.

In a further aspect the present invention provides the use of a compound of formula I in the manufacture of a medicament for the treatment and/or prophylaxis of conditions associated with a need for decreasing LDL-cholesterol levels.

In a further aspect the present invention provides the use of a compound of formula I in the manufacture of a medicament for the treatment and/or prophylaxis of inflammatory conditions.

In a further aspect the present invention provides the use of a compound of formula I in the manufacture of a medicament for the treatment and/or prophylaxis of Alzheimer's disease.

In a further aspect the present invention provides the use of a compound of formula I in the manufacture of a medicament for the treatment and/or prophylaxis of arteriosclerosis.

In a further aspect the present invention provides the use of a compound of formula I in the manufacture of a medicament for the treatment and/or prophylaxis of type 2 diabetes.

In a further aspect the present invention provides the use of a compound of formula I in the manufacture of a medicament for the treatment and/or prophylaxis of conditions associated with a need for improving HDL function.

In a further aspect the present invention provides the use of a compound of formula I in the manufacture of a medicament for the treatment and/or prophylaxis of hyperlipidemic conditions.

### Combination Therapy

The compounds of the invention may be combined with another therapeutic agent that is useful in the treatment of disorders associated with the development and progress of atherosclerosis such as hypertension, hyperlipidaernias, dyslipidaemias, diabetes, inflammation and obesity. The compounds of the invention may be combined with another therapeutic agent that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

In another aspect of the present invention, the compound of formula I, or a pharmaceutically acceptable salt or solvate thereof; or a solvate of such a salt, may be administered in association with cholesterol biosynthesis inhibitors, or pharmaceutically acceptable salts, solvates, solvates of such salts or prodrugs thereof. Suitable cholesterol biosynthesis inhibitors include HMG CoA reductase inhibitors, squalene synthesis inhibitors and squalene epoxidase inhibitors. A suitable squalene synthesis inhibitor is squalestatin 1 and a suitable squalene epoxidase inhibitor is NB-598. In this aspect of the invention, the compound of formula I, or a pharmaceutically acceptable salt or solvate thereof, or a solvate of such a salt, may be administrated in association with an HMG CoA reductase inhibitor, or pharmaceutically acceptable salts, solvates, solvates of such salts or prodrugs thereof. Suitably the HBG CoA reductase inhibitor, or pharmaceutically acceptable salts, solvates, solvates of such salts or prodrugs thereof are statins well known in the art. Particular statins are selected from the group consisting of atorvastatin, fluvastatin, pitavastatin, lovastatin, mevastatin, nicostatin, nivastatin, pravastatin and simvastatin, or a pharmaceutically acceptable salt, especially sodium or calcium, solvate, solvate of such a salt or a prodrug thereof. A particular statin is atorvastatin, or a pharmaceutically acceptable salt, solvate, solvate of such a salt or a prodrug thereof. A more particular statin is atorvastatin calcium salt. A particularly preferred statin is, however, rosuvastatin, or a pharmaceutically acceptable salt, solvate, solvate of such a salt or a prodrug thereof. A preferable particular statin is rosuvastatin calcium salt.

In the present application, the term “cholesterol biosynthesis inhibitors” also includes chemnical modifications of the HMG CoA reductase inhibitors, squalene synthesis inhibitors and squalene epoxidase inhibitors, such as esters, prodrugs and metabolites, whether active or inactive.

In another aspect of the present invention, the compound of formula I, or a pharmaceutically acceptable salt or solvate thereof, or a solvate of such a salt, may be administered in association with an inhibitor of the ileal bile acid transport system (IBAT inhibitor), or pharmaceutically acceptable salts, solvates, solvates of such salts or prodrugs thereof.

Suitable compounds possessing IBAT inhibitory activity have been described, see for instance the compounds described in WO 93/16055, WO 94/18183, WO 9.4/18184, WO 96/05188, WO 96/08484, WO 96/16051, WO 97/33882, WO 98/07449, WO 98/03818, WO 98/38182, WO 99/32478, WO 99/35135, WO98/40375, WO 99/35153, WO 99/64409, WO 99/64410, WO 00/01687, WO 00/47568, WO 00/61568, WO 00/62810, WO 01/68906, DE 19825804, WO 00/38725, WO 00/38726, WO 00/38727, WO 00/38728, WO 00/38729, WO 01/68906, WO 01/66533, WO 02/32428, WO 02/50051, EP 864 582, EP 489 423, EP 549 967, EP 573 848, EP 624 593, EP 624 594, EP 624 595 and EP 624 596 and the contents of these patent applications are incorporated herein by reference.

Further suitable compounds possessing IBAT inhibitory activity have been described in WO 94/24087, WO 98/56757, WO 00/20392, WO 00/20393, WO 00/20410, WO 00/20437, WO 01/34570, WO 00/35889, WO 01/68637, WO 02/08211, WO 03/020710, WO 03/022825, WO 03/022830, WO 03/022286, WO 03/091232, WO 03/106482, JP 10072371, U.S. Pat. No. 5,070,103, EP 251 315, EP 417 725, EP 869 121, EP 1 070 703 and EP 597 107 and the contents of these patent applications are incorporated herein by reference. Particular classes of IBAT inhibitors suitable for use in the present invention are benzothiazepines, and the compounds described in the claims, particularly claim 1, of WO 00/01687, WO 96/08484 and WO 97/33882 are incorporated herein by reference. Other suitable classes of IBAT inhibitors are the 1,2-benzothiazepines, 1,4-benzothiazepines and 1,5-benzothiazepines. A further suitable class of IBAT inhibitors is the 1,2,5-benzothiadiazepines.

One particular suitable compound possessing IBAT inhibitory activity is (3R,5R)-3-butyl-3-ethyl-1,1-dioxido-5-phenyl-2,3,4,5-tetrahydro-1,4-benzothiazepin-8-yl β-D-glucopyranosiduronic acid (EP 864 582). A further suitable compound possessing IBAT inhibitory activity is S-8921 (EP 597 107).

In another aspect of the present invention, the compound of formula I, or a pharmaceutically acceptable salt or solvate thereof, or a solvate of such a salt, may be administered in association with a cholesterol absorption antagonist, or pharmaceutically acceptable salts, solvates, solvates of such salts or prodrugs thereof, for example azetidinones such as ezetrol (zetia, ezetimibe) and those described in U.S. Pat. No. 5,767,115 which are incorporated herein by reference. Suitable compounds possessing cholesterol absorption antagonist activity have been described, see for instance the compounds described in WO 02/50027, WO 02/66464, WO 04/005247, WO 04/000803, WO 04/000804 and WO 04/000805 which are incorporated herein by reference.

In another aspect of the present invention, the compound of formula I, or a pharmaceutically acceptable salt or solvate thereof, or a solvate of such a salt, may be administered in association with a bile acid sequestrant or pharmaceutically acceptable salts, solvates, solvates of such salts or prodrugs thereof. Suitable bile acid sequestrants include cholestyramine, cholestipol and cosevelarn hydrochloride.

In another aspect of the invention, the compound of formula I, or a pharmaceutically acceptable salt or solvate thereof, or a solvate of such a salt, may be administered in association with a peroxisome proliferator-activated receptor (PPAR) modulating agent. PPAR modulating agents include but are not limited to a PPAR alpha and/or gamma and/or delta agonist, or pharmaceutically acceptable salts, solvates, solvates of such salts or prodrugs thereof. Suitable PPAR alpha and/or gamma and/or delta agonists, pharmaceutically acceptable salts, solvates, solvates of such salts or prodrugs thereof are well known in the art. These include the compounds described in WO 01/12187, WO 01/12612, WO 99/62870, WO 99/62872, WO 99/62871, WO 98/57941, WO 01/40170, WO 04/000790, WO 04/000295, WO 04/000294, WO 03/051822, WO 03/051821, WO 02/096863, WO 03/051826, WO 02/085844, WO 01/40172, J Med Chem, 1996, 39, 665, Expert Opinion on Therapeutic Patents, 10 (5), 623-634 (in particular the compounds described in the patent applications listed on page 634) and J Med Chem, 2000, 43, 527 which are all incorporated herein by reference. Particularly a PPAR alpha and/or gamma and/or delta agonist refers to muraglitazar (BMS 298585), rivoglitazone (CS-011), netoglitazone (MCC-555), balaglitazone (DRF-2593, N,N-2344), clofibrate, fenofibrate, bezafibrate, gemfibrozil, ciprofibrate, pioglitazone, rosiglitazone, AVE-0847, AVE-8134, CLX-0921, DRF-10945, DRF-4832, LY-518674, LY-818, LY-929, 641597, GW-590735, GW-677954, GW-501516, MBX-102, ONO-5129, KRP-101, R-483 (BM131258), TAK-559 or TAK-654. Particularly a PPAR alpha and/or gamma and/or delta agonist refers to tesaglitazar ((S)-2-ethoxy-3-[4-(2-{4-methanesulphonyl-oxyphenyl}ethoxy)phenyl]propanoic acid) and pharmaceutically acceptable salts thereof. In yet another aspect of the invention, the compound of formula I, or a pharmaceutically acceptable salt or solvate thereof, or a solvate of such a salt, may be administered in association with a pyruvate dehydrogenase kinase (PDK) inhibitor, or a pharmaceutically acceptable salt, solvate, solvate of such a salt or a prodrug thereof, or modulators of nuclear receptors such as retenoid X receptor (RXR), or a pharmaceutically acceptable salt, solvate, solvate of such a salt or a prodrug thereof.

In another aspect of the invention, the compound of formula I, or a pharmaceutically acceptable salt or solvate thereof, or a solvate of such a salt, may be administered in association with a cholesteryl ester transfer protein (CETP) inhibitor, or pharmaceutically acceptable salts, solvates, solvates of such salts or prodrugs thereof, for example those referenced and described in WO 00/38725 page 7 line 22-page 10, line 17 which are incorporated herein by reference.

In another aspect of the invention, the compound of formula I, or a pharmaceutically acceptable salt or solvate thereof, or a solvate of such a salt, may be administered in association with a microsomal transfer protein (MTP) inhibitor, or pharmaceutically acceptable salts, solvates, solvates of such salts or prodrugs thereof, for example implipatide and those described in WO 03/004020, WO 03/002533, WO 02/083658 and WO 00/242291, and the contents of these patent applications are incorporated herein by reference, or those described in Science, 282, 751-54, 1998 which are incorporated herein by reference.

In another aspect of the invention, the compound of formula I; or a pharmaceutically acceptable salt or solvate thereof, or a solvate of such a salt, may be administered in association with a nicotinic acid derivative, or pharmaceutically acceptable salts, solvates, solvates of such salts or prodrugs thereof, including slow release and combination products, for example, nicotinic acid (niacin), acipimox, nicofuranose, NIASPAN® and niceritrol.

In another aspect of the invention, the compound of formula I, or a pharmaceutically acceptable salt or solvate thereof, or a solvate of such a salt, may be administered in association with a acyl coenzymnA: cholesterol O-acyltransferase (ACAT) inhibitor, or pharmaceutically acceptable salts, solvates, solvates of such salts or prodrugs thereof, for example CS-505, eflucimibe (F-12511) and SMP-797.

In yet another aspect of the invention, the compound of formula I, or a pharmaceutically acceptable salt or solvate thereof, or a solvate of such a salt, may be administered in association with modulators of nuclear receptors such as farnesoid X receptor (FXR), or pharmaceutically acceptable salt, solvate, solvate of such a salt or a prodrug thereof.

In another aspect of the invention, the compound of formula I, or a pharmaceutically acceptable salt or solvate thereof, or a solvate of such a salt, may be administered in association with a phytosterol compound, or pharmaceutically acceptable salts, solvates, solvates of such salts or prodrugs thereof, for example stanols.

In another aspect of the invention, the compound of formula I, or a pharmaceutically acceptable salt or solvate thereof, or a solvate of such a salt, may be administered in association with other therapies for the treatment of metabolic syndrome or type 2 diabetes and its associated complications, these include biguanide drugs, for example metformin, phenformin and buformin, insulin (synthetic insulin analogues, amylin) and oral antihyperglycemics (these are divided into prandial glucose regulators and alpha-glucosidase inhibitors). An example of an alpha-glucosidase inhibitor is acarbose or voglibose or miglitol. An example of a prandial glucose regulator is repaglinide or nateglinide.

In another aspect of the invention, the compound of formula I, or a pharmaceutically acceptable salt or solvate thereof, or a solvate of such a salt, may be administered in association with a sulfonylurea for example: glimepiride, glibenclamide (glyburide), gliclazide, glipizide, gliquidone, chloropropamide, tolbutamide, acetohexainde, glycopyramide, carbutamide, glibonuride, glisoxepid, glybuthiazole, glibuzole, glyhexamide, glyrnidine, glypinamide, phenbutamide, tolcylamide and tolazamide. Preferably the sulfonylurea is glimepiride or glibenclamide (glyburide). More preferably the sulfonylurea is glimepiride. Therefore the present invention includes administration of a compound of the present invention in conjunction with one, two or more existing therapies described in this paragraph. The doses of the other existing therapies for the treatment of type 2 diabetes and its associated complications will be those known in the art and approved for use by regulatory bodies for example the FDA and may be found in the Orange Book published by the FDA. Alternatively smaller doses may be used as a result of the benefits derived from the combination.

In another aspect of the invention, the compound of formula I, or a pharmaceutically acceptable salt or solvate thereof, or a solvate of such a salt, may be administered in association with an antihypertensive compound for example an angiotensin converting enzyme (ACE) inhibitor, an angiotensin II receptor antagonist, an andrenergic blocker, an alpha andrenergic blocker, a beta andrenergic blocker, a mixed alphalbeta andrenergic blocker, an andrenergic stimulant, calcium channel blocker, an AT-1 blocker, a saluretic, a diuretic or a vasodilator, or pharmaceutically acceptable salts, solvates, solvates of such salts or prodrugs thereof. Particular ACE inhibitors or pharmaceutically acceptable salts, solvates, solvate of such salts or prodrugs thereof, including active metabolites, which can be used in combination with a compound of formula I include but are not limited to, the following compounds: alacepril, alatriopril, anrovenin, benazepril, benazepril hydrochloride, benazeprilat, benzoylcaptopril, captopril, captopril-cysteine, captopril-glutathione, ceranopril, cilazapril, cilazaprilat, delapril, delapril-diacid, enalapril, enalaprilat, enapril, epicaptopril, foroxymithine, fosfenopril, fosenopril, fosenopril sodium, fosinopril, fosinopril sodium, fosinoprilat, fosinoprilic acid, hemorphin-4, imidapril, indolapril, indolaprilat, lisinopril, lyciunin A, lyciumin B, moexipril, moexiprilat, muracein A, muracein B, muracein C, pentopril, perindopril, perindoprilat, pivalopril, pivopril, quinapril, quinapril hydrochloride, quinaprilat, ramipril, ramiprilat, spirapril, spirapril hydrochloride, spiraprilat, spiropril, spiropril hydrochloride, temocapril, temocapril hydrochloride, teprotide, trandolapril, trandolaprilat, zofenopril and zofenoprilat. Preferred ACE inhibitors for use in the present invention are ramipril, rarniprilat, lisinopril, enalapril and enalaprilat. More preferred ACE inhibitors for uses in the present invention are rarnipril and ramiprilat. Preferred angiotensin II receptor antagonists, pharmaceutically acceptable salts, solvates, solvate of such salts or a prodrugs thereof for use in combination with a compound of formula I include, but are not limited to, compounds: candesartan, candesartan cilexetil, losartan, valsartan, irbesartan, telmisartan and eprosartan. Particularly preferred angiotensin II receptor antagonists or pharmaceutically acceptable derivatives thereof for use in the present invention are candesartan and candesartan cilexetil.

In another aspect of the invention, the compound of formula I, or a pharmaceutically acceptable salt or solvate thereof, or a solvate of such a salt, may be administered in association with an anti-obesity compound, or pharmaceutically acceptable salts, solvates, solvates of such salts or prodrugs thereof, for example a pancreatic lipase inhibitor e.g. orlistat EP 129,748) or an appetite (satiety) controlling substance for example sibutramine (GB 2,184,122 and U.S. Pat. No. 4,929,629), a cannabinoid 1 (CB1) antagonist or inverse agonist, or pharmaceutically acceptable salts, solvates, solvates of such salts or prodrugs thereof, for example rimonabant (EP 656354) and as described in WO01/70700 or a melanin concentrating hormone (MCII) antagonist, or pharmaceutically acceptable salts, solvates, solvates of such salts or prodrugs thereof, for example as described in WO 04/004726.

In another aspect of the invention, the compounds of formula I, or a pharmaceutically acceptable salt or solvate thereof, or a solvate of such a salt, may be administrated in association with an anti-inflammatory agent such as glucocorticoids, non-steroidal anti-inflammatory agents (NSAID) or intestinal anti-inflammatory agents, or pharmaceutically acceptable salts, solvates, solvates of such salts or prodrugs thereof. Suitable glucocorticoids will include, but will not be limited to betametason, dexametason, methyl prednisolon, prednisolon, prednison, triamcinolon, hydrocortison, cortison and budesonid. Suitable non-steroidal anti-inflammatory agents will include, but will not be limited to indometacin, diklofenak, ibuprofen as well as acetylsalicylic acid. Suitable intestinal anti-inflammatory agents will include, but will not be limited to amino salicylates such as sulfasalazin, mesalazin, olsalazin and balsalazid.

In another aspect of the invention, the compounds of formula I, or a pharmaceutically acceptable salt or solvate thereof, or a solvate of such a salt, may be administrated in association with a cholinesterase inhibitor or an N-methyl-D-aspartate (NMDA) receptor antagonist, or pharmaceutically acceptable salts, solvates, solvates of such salts or prodrugs thereof, such as donepezil, rivastigmin or galantamin or memantin.

In an additional feature of the invention, there is provided a method for the treatment and/or prohylaxis of metabolic disorders in a warm-blooded animal, such as man, in need of such treatment which comprises administering to said animal an effective amount of a compound of formula I, or a pharmaceutically acceptable salt or solvate thereof, or a solvate of such a salt, in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of dyslipidemia in a warm-blooded animal, such as man, in need of such treatment which comprises administering to said animal an effective amount of a compound of formula I, or a pharmaceutically acceptable salt or solvate thereof, or a solvate of such a salt, in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of the insulin resistance syndrome in a warm-blooded animal, such as man, in need of such treatment which comprises administering to said animal an effective amount of a compound of formula I, or a pharmaceutically acceptable salt or solvate thereof, or a solvate of such a salt, in simultaneous, sequential or separate administration with an effective amount of one of the other compounds described in this combination section, or a pharmaceutically acceptable salt, solvate, solvate of such a salt or a prodrug thereof.

Therefore in an additional feature of the invention, there is provided a method for the treatment and/or prophylaxis of type 2 diabetes and its associated complications in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

Therefore in an additional feature of the invention, there is provided a method of treating and/or preventing hyperlipidemic conditions in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section or a pharmaceutically acceptable salt, solvate, solvate of such a salt or a prodrug thereof.

In an additional feature of the invention, there is provided a method for the treatment and/or prohylaxis of cardiovascular disease in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of atherosclerosis in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of hypercholesterolemia in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for treating and/or preventing conditions associated with a need for improving reverse cholesterol transport in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for treating and/or preventing conditions associated with a need for decreasing intestinal cholesterol absorption in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for treating and/or preventing conditions associated with a need for increasing HDL-cholesterol levels in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for treating and/or preventing conditions associated with a need for decreasing LDL-cholesterol levels in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of inflammatory conditions in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of Alzheimer's disease in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for the treatment and/or prohylaxis of arteriosclerosis a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

In an additional feature of the invention, there is provided a method for treating and/or preventing conditions associated with a need for improving HDL function in a warm-blooded animal, such as man, in need of such treatment which comprises administering to said animal an effective amount of a compound of formula I, or a pharmaceutically acceptable salt or solvate thereof, or a solvate of such a salt in simultaneous, sequential or separate administration with an effective amount of one of the other compounds described in this combination section, or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt or solvate thereof, or a solvate of such a salt, and one of the other compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt or solvate thereof, or a solvate of such a salt, and one of the other compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt or solvate thereof, or a solvate of such a salt, in a first unit dosage form;
b) one of the other compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt or solvate thereof, or a solvate of such a salt, together with a pharmaceutically acceptable diluent or carrier, in a first unit dosage form;
b) one of the other compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of metabolic disorders in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of dyslipidemia in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of metabolic syndrome or type 2 diabetes and its associated complications in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of hyperlipidaemic conditions in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of cardiovascular disease in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of atherosclerosis in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of hypercholesterolemia in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of a conditions associated with a need for improving reverse cholesterol transport in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of a conditions associated with a need for decreasing intestinal cholesterol absorption in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of a conditions associated with a need for increasing HDL-cholesterol levels in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of a conditions associated with a need for decreasing LDL-cholesterol levels in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a is pharmaceutically acceptable salt, solvate, solvate of such a salt or a prodrug thereof, in the manufacture of a medicament for use in the treatment and/or prophylaxis of inflammatory conditions in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of Alzheimer's disease in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of arteriosclerosis in a warm-blooded animal, such as man.

According to another feature of the invention there is provided the use of a compound of the formula I, or a pharmaceutically acceptable salt or solvate thereof, or a solvate of such a salt, and one of the other compounds described in this combination section, or a pharmaceutically acceptable salt, solvate, solvate of such a salt or a prodrug thereof, in the manufacture of a medicament for use in the treatment and/or prophylaxis of a conditions associated with a need for improving HDL function in a warm-blooded animal, such as man.

According to a further aspect of the present invention there is provided a combination treatment comprising the administration of an effective amount of a compound of the formula I, or a pharmaceutically acceptable salt or solvate thereof, or a solvate of such a salt, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

### EXAMPLES

### Abbreviations

DCM Dichloromethane
DMF N,N-dimethylformamide
DMSO dimethylsulfoxide
EtOAc ethyl acetate
EtOH ethanol
HPLC high performance liquid chromatography
NMR nuclear magnetic resonance
TFA trifluoroacetic acid
THF tetrahydrofuran
UV ultra violet
h hour
min. minutes
rt room temperature
br broad
bs broad singlet
bt broad triplet
d doublet
dd doublet of doublets
m multiplet
q quartet
singlet
t triplet

### General Experimental Procedures

Flash column chromatography employed normal phase silica gel 60 (0.040-0.0063 mm, Merck) or IST Isolute®SPE columns normal phase silica gel. Purifications were performed is on either a Gilson preparative HPLC system with a UV triggered fraction collector, equipped with a ACE C8 5 μm 250 mm×20 mm column, or on a Waters preparative HPLC system equipped with an ACE C8 5 μm 250 mm×50 mm column or an ACE C8 5 μm 250 mm×20 mm column. ¹H NMR spectra were obtained on a Varian Unity Plus, 400 M1 Hz, operating at 9.3 T, equipped with a 5 mm switchable probe with an inner X-coil, for solutions in CDCl₃ (residual CHCl₃ (δ_{H} 7.23 ppm) as internal standard), or DMSO-d (residual DMSO (BH 2.50 ppm) as internal standard) at 300K. Chemical shifts are given in ppm. Microwave heating was performed using single node heating in a Smith Creator from Personal Chemistry, Uppsala, Sweden. Lawesson's reagent is 2,4-Bis(4-methoxyphenyl)-1,3,2,4-dithiadiphosphetane-2,4-disulfide.

### Synthesis of Starting Materials and Intermediates

### 3-Hydroxy-4-phenyl-1H-pyrrole-2,5-dione

Prepared according to literature procedure: C. S. Rooney, et al; J. Med. Chem., Vol. 26 (1983) pp 700-714.

### 3-Chloro-4-phenyl-1H-pyrrole-2,5-dione

To a suspension of 3-hydroxy-4-phenyl-1H-pyrrole-2,5-dione (25.0 g, 0.13 mol) in dichloromethane (600 mL) under an atmosphere of nitrogen was added DMF (36 mL). The suspension was cooled to ice temperature and treated with oxalyl chloride (40.0 g, 0.32 mol). The reaction mixture was subsequently refluxed overnight. After cooling to rt, silica gel was added and the reaction mixture evaporated to dryness and subjected to flash chromatography (hexane:EtOAc, 80:20). Trituration with dichloromethane, filtration and drying gave (17.6 g, 64%) of the title compound; ¹H NMR (400 Maz, CDCl₃) δ 7.96-7.89 (m, 2H), 7.88-7.77 (bs, 1H), 7.55-7.45 (m, 3H).

### tert-butyl [5-(bromomethyl)pyridin-2-yl]carbamate

Prepared according to literature procedure: WO0066557 Linschoten, M. et al, Astrazeneca AB, Nov. 9, 2000.

### tert-Butyl {5-[(3-chloro-2,5-dioxo-4-phenyl-2,5-dihydro-1H-pyrrol-1-yl)methyl]pyridin-2-yl}carbamate

3-Chloro-4-phenyl-1H-pyrrole-2,5-dione (1.55 g, 7.47 mmol) was dissolved in DMF (25 mL) under nitrogen atmosphere and cooled in an ice-bath. tert-Butyl [5-(bromomethyl)pyridin-2-yl]carbamate (2.14 g, 7.47 mmol) was added followed by anhydrous potassium carbonate (1.03 g, 7.47 mmol). The mixture was stirred for 1.5 h whereafter the cooling-bath was removed and the mixture was stirred for another two h and then neutralized with 1% HCl. Water (100 mL) was added and the mixture was extracted with CH₂Cl₂ (50 mL×3). The extracts were combined, washed with water (100 mL×2), dried with magnesium sulphate, filtered and evaporated. The crude product (3.41 g) was used in the next step without further purification; ¹H NMR (400 Mz, CDCl₃) δ 8.32 (d, J=2 Hz, 1H), 7.92-7.89 (m, 3H), 7.83 (bs, 1H), 7.72 (dd, J=9, 2 Hz, 1H), 7.49-7.47 (m, 3H), 4.71 (s, 2H) and 1.52 (s, 9H).

### 1-[(6-Aminopyridin-3-yl)methyl]-3-{[4-(difluoromethoxy)phenyl]amino}-4-phenyl-1H-pyrrole-2,5-dione

A mixture of tert-butyl {5-[(3-chloro-2,5-dioxo-4-phenyl-2,5-dihydro-1H-pyrrol-1-yl)methyl]pyridin-2-yl}carbamate (0.70 g, 1.7 mmol) and 4-(difluoromethoxy)aniline (0.54 g, 3.4 mmol) in DMF (4 mL) was heated in a microwave reactor at 150° C. for 8 minutes. The solvent was evaporated and the residue was purified on a pre-packed SiO₂ column (Isolute® SI, 10 g/70 mL) using CH₂Cl₂ and then CH₃OH/CH₂Cl₂ (1:99, 2:98 and then 5:95) as eluant to give 0.4 g (54%) of the title compound; ¹H NMR (400 MHz, CDCl₃) δ 7.99 (bs, 1H), 7.67-7.62 (m, 2H), 7.14-7.04 (m, 3H), 6.91 (d, J=8 Hz, 2H), 6.78 (d, J=8 Hz, 1H), 6.72 (d, J=9 Hz, 2H), 6.63 (d, J=9 Hz, 2H), 6.33 (t, J=74 Hz, 1H) and 4.60 (s, 2H).

### Examples

### Example 1

### 1-[(6-aminopyridin-3-yl)methyl]-3-{[4-(difluoromethoxy)phenyl]amino}-4-phenyl-5-thioxo-1,5-dihydro-2H-pyrrol-2-one

Phosphorus pentasulfide (1.6 g, 3.6 mmol) was added into 1-[(6-aminopyridin-3-yl)methyl]-3-{[4-(difluoromethoxy)phenyl]amino)}-4-phenyl-1H-pyrrole-2,5-dione (2.83 g, 6.5 mmol) in dioxane (100 mL). It was dipped into a preheated oil bath at 120° C. The reaction mixture was refluxed for 15 minutes, cooled to room temperature and concentrated. The residue was purified by preparative HPLC using acetonitrile/water (0.1% TFA) system. The acetonitrile was removed in vacuo at room temperature and it was then freeze dried. The obtained material was dissolved in DCM (10 mL). It was then shaken with a mixture of NaHCO₃ (2 mL, sat.) and brine (1 mL) in a separatory funnel. The two phases were separated and the organic phase was dried (Na₂SO₄) and evaporated. It was further purified by column chromatography (ISOLUTE SI, 5 g/25 mL), eluting with DCM, MeOH:DCM (0.5: 99.5, then 1:99), to give the (2 mg) of the title compound; ¹H NMR (400 MHz, CDCl₃): [symbol] 4.59 (br, 2H), 5.04 (s, 2H), 6.32 (t, 1H), 6.46 (d, 1H), 6.60 (d, 2H), 6.69 (d, 2H), 6.93-6.96 (m, 3H), 7.06-7.14 (m, 3H), 7.60 (dd, 1H), 8.19 (d, 2H); MS: M−H⁺451.

### Biological Activity

### Co-Activator Recruitment Assay

The Ligand Binding Domain (LBD) of human LXRalpha (amino acid 205-447) and LXRbeta (amino acid 216-461) was produced by recombinant techniques in E. coli. A fragment of the human Steroid Receptor Co-Activator-1 (SRC-1) was produced as a synthetic peptide. An anti-6His-antibody coupled with Europium Eu³⁺) was used to recognize the His-tag on the LXR-LBD and Allophycocyanin (APC) coupled to streptavidin was used to recognize the biotinylated SRC-1. Agonist binding to LXRalpha or LXRbeta enhances the affinity of LXR towards SRC-1 and thereby brings Eu³⁺ and APC in close proximity. Eu³⁺ is excited at 337 nm and emitts light at 620 nm. This emission, when in close proximity, excites APC to emit light at 665 nm.

Dilution plates with compounds in DMSO were further diluted in buffer {20 mM [Tris(hydroxymethyl)aminomethane] pH 7.5, 0.125% CHAPS {3-[(3-Cholamidopropyl)dimethylammonio]-1-propanesulfonate}, 2 mM DTT (Dithiothreitol) and 0.05% BSA (Bovine Serum Albumin)} in order to reduce DMSO concentration, 0.5 μl to 13.5 μl. To this, 6 μl assay mix was added and the plates (384-well V-groove plates) were incubated at room temperature for 60 to 80 min. The assay mix has the following final concentrations; LXRalpha mix: 0.06 μg/mL Eu-labelled anti-6×His Ab, 1.15 μg/mL Streptavidin APC, 30 nM SRC-1 peptide and 0.9 μg/mL LXRalpha in buffer and LXRbeta mix; 0.06 μg/mL Eu-labelled anti-6×His Ab, 1.15 μg/mL Streptavidin APC, 90 nM SRC-1 peptide and 0.2 μg/mL LXRbeta in buffer. Time-resolved fluorescence readings were done in a Wallac Victor reader at 665 nm followed by reading at 615 nm. The LXR ligand, 22-R Hydroxycholesterol at 50 μM was used as the 100% control.

### Transactivation Assay

Expression vectors were prepared by inserting the ligand binding domain cDNA (complementary DNA) of human LXRalpha (amino acid 205447) and LXRbeta (amino acid 216-461) in frame with, 3′ to the yeast GAL4 transcription factor DNA binding domain and the nuclear localization signal from the T-antigen of Polyoma Virus in the eucaryotic expression vector pSG5 (Stratagene). The resulting expression vectors pSGGAL-LXRalpha and pSGGAL-LXRbeta were used in cotransfection experiments together with the pGL3 luciferase reporter plasmid containing a minirnal SV40 promoter and five copies of the UAS GAL4 recognition site. 2.5 μg pSGGAL-LXRalpha or beta were mixed with 25 μg pGL3 5×UAS and 22.5 μg pBluscript in 0.95 mL ice cold PBS containing approx. 4-9 milj. U2/OS osteosarcoma cells. After a five minute incubation on ice the cell/DNA mixture was electroporated in 0.4 cm cuvettes at 960 μF, 230 V using a BioRad electroporator and diluted to 0.32 milj cells/mL in complete DMEM (Dulbecco's Modified Eagle Medium) medium (Gibco 31966-021). Cells from at least two electroporations were pooled in order to avoid variations between different electroportations. 25 μl diluted, electroporated cells, were seeded onto 384-well plates (0.8×1 cells/well) and the cells were allowed to adhere for 2 h at 37° C., 5% CO₂ in a cell culture incubator. Dilution plates with compounds in DMSO were further diluted in DMEM w/o phenol red (Gibco 11880-028) including 10% FBS (Foetal Bovine Serum), 1% PEST (Penicillin Streptomycin), 20 mM Hepes, 2 mM L-Glutamine and 0.36% Glucose (2.5 μl to 97.5 μl) in order to reduce DMSO concentration. 7 μl of this was added to the electroporated cells in 384-well plates and incubation was continued for 48 h in a cell culture incubator, after which cells were lysed by adding 32 μl/well LucLite luciferase substrate. Luciferase activity was measured using the “Luminescence 384 protocol” in the Wallac Victor reader after 15 min. incubation at room temperature. The LXR ligand, Tularik T0901317, at 1 μM was used as the 100% control.

The compounds of formula I have an EC₅₀ of less than 50 μmol/l for LXRalpha and/or beta in coactivator recruitment assays and/or reporter gene assays. For example, the compound of Example 1 was EC₅₀'s of 0.28 μmol/l and 0.32 μmol/l in reporter gene assays, respectively.

In addition the compounds of the present invention exhibit improved physical and/or chemical and/or DMPK (Drug Metabolism and Pharmacokinetic) properties, for example they exhibit improved metabolic stability in vitro, and/or exhibit favourable pharmacological effects in vivo. The compounds also have a promising toxicological profile.

1. A compound of Formula I 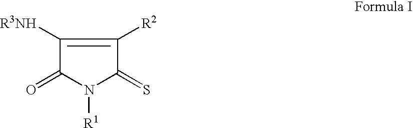
wherein:
R¹ is selected from phenyl(1-4C)alkyl wherein the phenyl is optionally substituted by (1-4C)alkoxycarbonyl or a group of formula NR^{a}R^{b} in which R^{a} and R^{b} independently represent H or (1-4C)alkyl; heteroaryl(1-4C)alkyl wherein the heteroaryl is optionally substituted by (1-4C)alkyl or a group of formula NR^{a}R^{b} in which R^{a} and R^{b} independently represent H or (1-4C)alkyl; or a (1-6C)alkyl group which is optionally substituted by one or more of the following: fluoro, (1-4C)alkoxycarbonyl, (1-3C)alkylthio or (1-3C)alkoxy optionally substituted by one or more fluoro;
R² is phenyl; and
R³ is selected from phenyl, indolyl or benzofuranyl each optionally substituted by one or more of the following: (1-3C)alkanoyl, (1-4C)alkoxy optionally substituted by one or more fluoro; (1-3C)alkylthio; or a group of formula NR^{a}R^{b} in which R^{a} and R^{b} independently represent H, (1-3C)alkyl or (1-3C)alkanoyl or R^{a} and R^{b} together with the nitrogen atom to which they are attached represent morpholino;
or a pharmaceutically acceptable salt or solvate thereof, or a solvate of such a salt.

2. A compound according to claim 1 in which R¹ is selected from methyl, ethyl, propyl, butyl, 2-methoxyethyl, 2,2,2-trifluoroethyl, benzyl, 4-pyridylmethyl, 3-pyridylmethyl or 6-amino-3-pyridylmethyl.

3. A compound according to claim 1 in which R³ is 4-methoxyphenyl, 4-difluoromethoxyphenyl or 4-morpholinophenyl.

4. A compound according to claim 1 wherein R¹ is selected from methyl, ethyl, 2,2,2-trifluoroethyl, benzyl, 3-pyridylmethyl or 6-amino-3-pyridylmethyl;
R² is phenyl; and
R³ is selected from 4-methoxyphenyl, 4-difluoromethoxyphenyl or 4-morpholinophenyl.

5. A compound according to claim 1 wherein R¹ is selected from ethyl, 2,2,2-trifluoroethyl, benzyl, 3-pyridylmethyl or 6-amino-3-pyridylmethyl;
R² is phenyl; and
R³ is selected from 4-methoxyphenyl, 4-difluoromethoxyphenyl or 4-morpholinophenyl.

6. A compound according to claim 1 wherein R¹ is selected from methyl, ethyl, 2,2,2-trifluoroethyl, 2-pyridylmethyl, 3-pyridylmethyl or 4-pyridylmethyl;
R² is phenyl; and
R³ is 4-methoxyphenyl.

7. A compound according to claim 1 wherein R¹ is 2-methoxyethyl or 6-amino-3-pyridylmethyl;
R² is phenyl; and
R³ is 4-methoxyphenyl or 4-difluoromethoxyphenyl.

8. 1-[(6-Aminopyridin-3-yl)methyl]-3-{[4-(difluoromethoxy)phenyl]amino}-4-phenyl-5-thioxo-1,5-dihydro-2H-pyrrol-2-one or a pharmaceutically acceptable salt or solvate thereof, or a solvate of such a salt.

9. A process for the preparation of a compound according to claim 1, comprising reacting a compound of formula II, 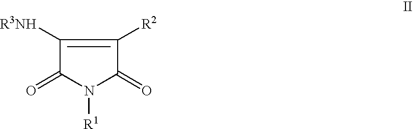
wherein R² and R³ are as defined in claim 1, with a sulphurating agent, optionally in the presence of an inert organic liquid at a temperature in the range of 0° C. to 200° C.

10. A pharmaceutical formulation comprising a compound according to claim 1 and a pharmaceutically acceptable adjuvant, diluent and/or carrier.

11-25. (canceled)

26. A method of treating and/or preventing lipid disorders (dyslipidemia) whether or not associated with insulin resistance comprising the administration of a compound according to claim 1 to a mammal in need thereof.

27. A method for treatment and/or prophylaxis of cardiovascular disease comprising administering to a mammal, including man, in need of such a treatment an effective amount of a compound as defined in claim 1.

28. A method of treating and/or preventing atherosclerosis comprising the administration of an effective amount of a compound of formula I according to claim 1 to a mammal in need thereof.

29. A method for treatment and/or prophylaxis of hypercholesterolemia comprising administering to a mammal, including man, in need of such a treatment an effective amount of a compound as defined in claim 1.

30. A method for treatment and/or prophylaxis of a condition associated with a need for improving reverse cholesterol transport comprising administering to a mammal, including man, in need of such a treatment an effective amount of a compound as defined in claim 1.

31. A method for treatment and/or prophylaxis of a condition associated with a need for decreasing intestinal cholesterol absorption comprising administering to a mammal, including man, in need of such a treatment an effective amount of a compound as defined in claim 1.

32. A method for treatment and/or prophylaxis of a condition associated with a need for increasing HDL-cholesterol levels comprising administering to a mammal, including man, in need of such a treatment an effective amount of a compound as defined in claim 1.

33. A method for treatment and/or prophylaxis of a condition associated with a need for decreasing LDL-cholesterol levels comprising administering to a mammal, including man, in need of such a treatment an effective amount of a compound as defined in claim 1.

34. A method for treatment and/or prophylaxis of inflammatory a condition comprising administering to a mammal, including man, in need of such a treatment an effective amount of a compound as defined in claim 1.

35. A method for treatment and/or prophylaxis of Alzheimer's disease comprising administering to a mammal, including man, in need of such a treatment an effective amount of a compound as defined in claim 1.

36. A method for treatment and/or prophylaxis of arteriosclerosis comprising administering to a mammal, including man, in need of such a treatment an effective amount of a compound as defined in claim 1.

37. A method for treatment and/or prophylaxis of type 2 diabetes comprising administering to a mammal, including man, in need of such a treatment an effective amount of a compound as defined in claim 1.

38. A method for treatment and/or prophylaxis of a condition associated with a need for improving HDL function comprising administering to a mammal, including man, in need of such a treatment an effective amount of a compound as defined in claim 1.

39. (canceled)

40. A pharmaceutical composition comprising a compound as claimed in any claim 1 and another therapeutic agent that is useful in the treatment of a condition or disorder associated with the development and progress of atherosclerosis or a condition associated with a need for improving reverse cholesterol transport and/or decreasing intestinal cholesterol absorption.

41. A process of claim 9 wherein the sulphurating agent is Lawesson's reagent.

42. A process of claim 9 wherein the inert organic liquid is an aromatic hydrocarbon.

43. A process of claim 42 wherein the aromatic hydrocarbon is toluene.

44. A pharmaceutical composition according to claim 40 wherein the condition or disorder associated with the development and progress of atherosclerosis is hypertension, dyslipidemias, hyperlipidaemias, hypercholesterolemias, type 2 diabetes, inflammation, or obesity as well as conditions associated with a need for improving reverse cholesterol transport and/or decreasing intestinal cholesterol absorption.
